(19) United States
(12) Patent Application Publication
(10) Pub. No.: US 2013/0131541 A1
(43) Pub. Date: May 23, 2013

(54) CELL PHONE APP FOR COUPLING A CELL PHONE TO A BASAL BODY TEMPERATURE THERMOMETER FOR PREDICTING OVULATION

(71) Applicants: Hwai Sian Tsai, Tsuen Wan (HK); Suk Kuen Wong, Tsuen Wan (HK)

(72) Inventors: Hwai Sian Tsai, Tsuen Wan (HK); Suk Kuen Wong, Tsuen Wan (HK)

(73) Assignee: Eternal Electronics Limited, Tsuen Wan (HK)

(21) Appl. No.: 13/303,663

(22) Filed: Nov. 23, 2011

(51) Int. Cl. A61B 10/00

(52) U.S. Cl. 600/551

(57) ABSTRACT

A system for predicting ovulation cycles by employing an application running on a mobile communication device such as an iPhone®, a Blackberry™, or an Android-based smartphone is provided. The system includes a basal body temperature thermometer coupled to a mobile communication device. The thermometer takes temperature measurements, encodes the temperature measurements and transmits the encoded temperature measurements to the mobile communication device. The mobile communication device receives and decodes the encoded temperature measurements. The mobile communication device includes an application that analyzes the decoded temperature measurements to determine whether they are satisfactory or unsatisfactory, provides feedback to a user in real-time and provides an ovulation prediction.

### FIELD OF THE INVENTION

The present invention relates to an ovulation prediction system, and, more particularly, to a system for predicting ovulation by employing an application running on a mobile communication device such as an iPhone®, a Blackberry™ or an Android-based smartphone, a BBT thermometer and online database and tools.

### BACKGROUND OF THE INVENTION

Basal Body Temperature (BBT) charting is a well-known and widely used method for predicting ovulation. BBT is generally measured with a BBT thermometer such as the one disclosed in U.S. Pat. No. 4,377,171 to Wada. BBT is the lowest temperature attained by the body during rest. It is generally measured immediately after awakening and before any physical activity has been undertaken. Moreover, it is advised that the BBT be measured before a user gets up or even speaks. In other words, the most optimal results can generally be achieved when the BBT is taken the moment the user wakes up. In women, ovulation causes an increase of approximately one-half to one degree Fahrenheit (approximately one-quarter to one-half degree Celsius) in basal body temperature (BBT).

Monitoring of BBTs is one of the ways of estimating a woman's most fertile time of the month. To estimate this, a woman must determine the time of ovulation and the pattern of her reproductive cycle. This may be attained by taking BBT readings first thing every morning for several months and recording the measured readings along with other supplementary information such as cervical mucus observations and menstrual cycle data. This information is usually plotted by hand on graph paper or using computer software to better illustrate the changes that occur during the reproductive cycle. The charts are then interpreted to determine when ovulation occurred over the last several months and to predict when it is likely to occur in the future. Charting software and applications are well-known in the art and available from companies such as OvuSoft LLC, FertilityFriend.com and Clocking Edge, LLC. Such programs allow a user to manually input BBT data and other supplementary data and are able to provide an ovulation prediction after several weeks of consistent use.

As noted above, in order to collect reliable BBT data, a user needs to measure BBT routinely, at the same time every day. However, in addition to that, the BBT also needs to be properly and accurately measured and then accurately recorded and plotted. Moreover, due to the nature of BBT readings and the fact that they must be taken at the same time every day, unless such readings are recorded and analyzed immediately, erroneous readings may not be discovered until it is too late to retake a reading. It often is too time consuming or inconvenient to immediately input the readings into the computer and generate graphs. Moreover, reference to these records or charts may be inconvenient for a user.

This is especially problematic for users with busy schedules. As indicated above, the BBT measurement has to be taken every day, at the same time. In other words, if an average user wakes up at 7:00 am, she will have to take the measurement at that time, even on weekends or holidays. A time difference of about 30 minutes should not make drastic changes in accuracy of BBT measurements, thus, a user might be able to measure the BBT accurately from approximately 6:30 am to 7:30 am. However, on certain days a user might wake up late and take a BBT measurement too late, or a user might forget to take a measurement at all, or a user might take the measurement at a proper time, but might forget to record it, or record it incorrectly. Additionally, a user might record the BBT reading provided by her thermometer, but she may not have the time to analyze it to determine whether there is something wrong with the reading. The user in that case would likely record the incorrect reading, which might throw off all the other data, leading to inaccurate ovulation cycle predictions. Thus, an inaccurate reading reported during just one day may lead to unusable results for the entire cycle. Since even very small changes in BBT measurements make a very significant difference, it is critical that BBT measurements are recorded and recorded correctly every day.

Accordingly, there is a need for a simple and cost-effective system for providing a personal ovulation predictor that is reliable and convenient to use. There is a need for a device that can (1) remind the user every day, at the same time, to take the BBT reading, (2) accurately record such reading, (3) analyze such a reading and alert the user if the reading seems inaccurate for any reason, (4) plot such a reading on an ovulation chart, and (5) make accurate ovulation predictions.

### SUMMARY OF THE INVENTION

The proposed system for predicting ovulation cycles employs an application running on a mobile communication device such as a smartphone, which may be an iPhone®, a Blackberry™, or an Android-based smartphone. The system includes a basal body temperature thermometer coupled to a mobile communication device. The thermometer takes temperature measurements, encodes the temperature measurements and transmits the encoded temperature measurements to the mobile communication device. The mobile communication device receives and decodes the encoded temperature measurements. The mobile communication device includes an application that analyzes the decoded temperature measurements to determine whether they are satisfactory or unsatisfactory and provides feedback to a user in real-time. The application additionally provides an ovulation prediction. The proposed system eliminates the requirement that a user manually record the basal body temperature data, which results in significant time savings and leads to higher accuracy.

In one exemplary embodiment, the thermometer includes a microcontroller unit and a dual-tone multi-frequency (DTMF) tone generator. The microcontroller enables the DTMF tone generator to encode or modulate the temperature measurements by generating dual-tone signals corresponding to those temperature measurements. The encoded or modulated measurements are then transmitted to the mobile communication device, which decodes or demodulates the measurements. The application then displays the temperature measurements as numeric characters, records them, analyzes them and provides feedback to the user in real-time. The feedback may include, inter alia, a suggestion that the user retake the temperature measurement immediately or that the user take a short break and then retake the temperature measurement.

In another embodiment, the application reminds a user to take her basal body temperature at the same time every day to achieve accurate basal body temperature measurements. In yet another embodiment, the application asks the user a series of questions if it determines, based on predetermined characteristics, that the reading is inaccurate or inconsistent. The reading may be characterized as inaccurate if it does not conform with the trend of the readings for a predetermined period prior to the day that the temperature measurement is taken. The reading may also be characterized as inaccurate if the application determines, based on a set of predetermined characteristics, that the reading is inconsistent with the temperature measurement on the same day of the previous ovulation cycle or cycles.

The foregoing and additional aspects of the present invention will be apparent to those of ordinary skill in the art in view of the detailed description of various embodiments, which is made with reference to the drawings, a brief description of which is provided next.

### BRIEF DESCRIPTION OF THE DRAWINGS

The foregoing and other advantages of the invention will become apparent upon reading the following detailed description and upon reference to the drawings. Embodiments of the present invention will now be described, by way of example only, with reference to the attached Figures.

FIG. 1 is a diagram of a mobile communication device connected to a BBT thermometer using a plug, on-line database and tools.

FIG. 2 is a diagram of the BBT thermometer communicating with the mobile communication device through DTMF tones, on-line database and tools.

FIG. 3 is a diagram of the BBT thermometer communicating with the mobile communication device through a Bluetooth or Wi-Fi connection, on-line database and tools.

### DETAILED DESCRIPTION OF THE ILLUSTRATED EMBODIMENTS

Although the invention will be described in connection with certain preferred embodiments, it will be understood that the invention is not limited to those particular embodiments. On the contrary, the invention is intended to cover all alternatives, modifications, and equivalent arrangements as may be included within the spirit and scope of the invention as defined by the appended claims.

This invention is directed to a system for predicting ovulation using a mobile communication device application (APP) and a BBT thermometer in communication with the APP. A mobile communication device may be a smartphone. The smartphone may be an iPhone®, a Blackberry™ or an Android-based phone. To enable communication between the thermometer and the APP, the thermometer is equipped with a dual-tone multi-frequency (DTMF) tone generator or any other signal encoder or modulator that outputs an encoded or modulated signal compatible with the circuitry of a mobile communication device. Alternatively, the BBT thermometer may be equipped with Bluetooth, Wi-Fi or any other near-field communication technology that allows direct communication between the thermometer and the APP. Another alternative embodiment involves equipping the BBT thermometer with a connector, such as a tip-ring-sleeve (TRS) connector, a 3.5 mm plug, a 2.5 mm plug, or any other connector that is adapted to fit into an earphone/microphone input port of a mobile communication device. The connector is plugged into a corresponding port of the mobile communication device to enable communication between the thermometer and the APP and send encoded or modulated signals via the audio channel/microphone input port.

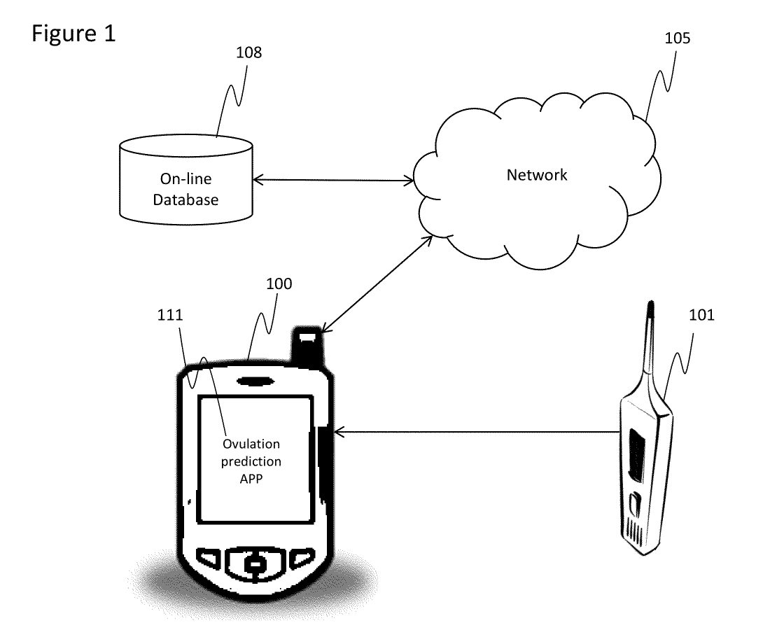

Referring now to FIG. 1, a BBT thermometer 101 is in communication with a mobile communication device 100, which has an APP 111 for predicting ovulation. The APP 111 can communicate to a database or web application 108 via one or more networks 105.

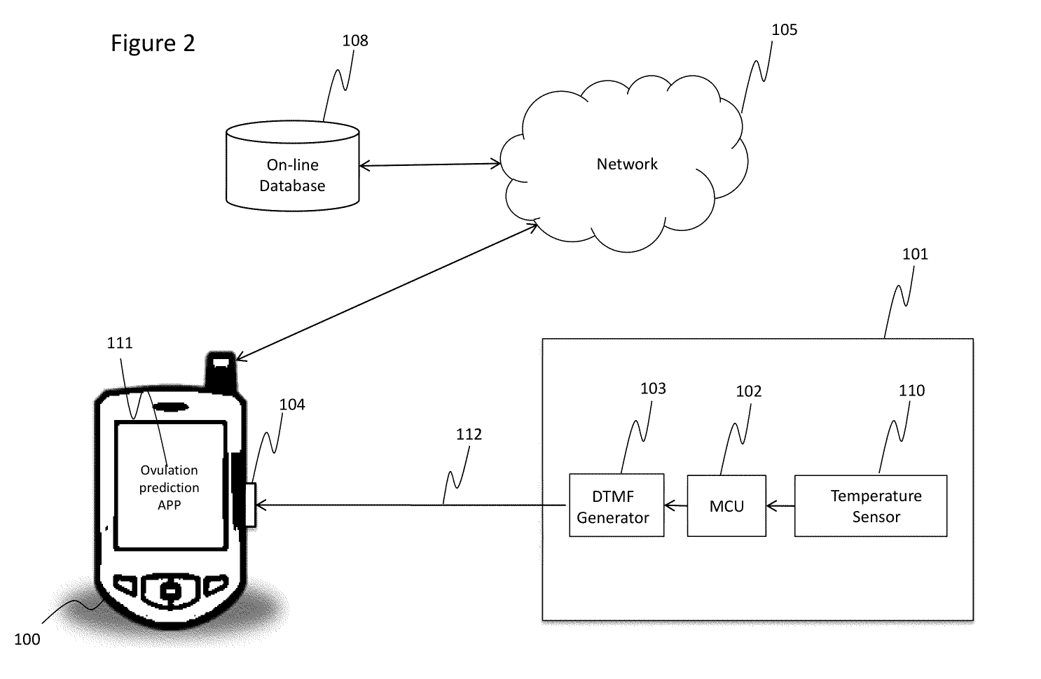

FIG. 2 depicts one embodiment where the BBT thermometer 101 is equipped with a connector 112 adapted to fit into an earphone/microphone input port 104 of the mobile communication device 100, and a temperature sensor 110. The temperature sensor 110 produces an electrical signal that varies according to the sensed temperature and provides temperature readings. The BBT thermometer 101 is connected to the mobile communication device 100 via the input port 104. It is envisioned, however, that the BBT thermometer 101 may be coupled to the mobile communication device 100 through other available ports of the mobile communication device 100 as well, and those skilled in the art will appreciate that the claimed invention can work with any connector or connector configuration that is available.

In this case, the BBT thermometer 101 is equipped with a microcontroller unit (MCU) 102 and a dual-tone multi-frequency (DTMF) tone generator 103, which may be a buzzer. The MCU 102 has stored therein a set of instructions that enable the DTMF generator to generate dual-tone signals corresponding to the temperature measurements. Once the user takes a BBT measurement and once the thermometer 101 is connected to the mobile communication device 100, the MCU 102 enables the DTMF tone generator 103 to generate a dual tone corresponding to the BBT measurement and thereby encode the temperature measurement into a dual-tone sinusoidal signal. According to the DTMF coding scheme, a combination of frequencies are used to form a table, for example:

TABLE 1

| | f5 | f6 | f7 | f8 |
|---|---|---|---|---|
| f1 | 0 | 1 | 2 | 3 |
| f2 | 4 | 5 | 6 | 7 |
| f3 | 8 | 9 | A | B |
| f4 | C | D | E | F |

The user takes a BBT measurement by using the BBT thermometer 101 for a pre-determined amount of time. The thermometer 101 may optionally provide a timer to indicate that the measurement is complete. Once the measurement is complete, the thermometer 101 generates sounds in different dual tones generated by the DTMF generator 103 to represent the temperature reading. The thermometer 101 measures and represents the temperature in numerical digits in Celsius scale or Fahrenheit scale, for example:

38.13 degrees Celsius or

100.63 degrees Fahrenheit.

For the purpose of human body temperature measurement, it is very safe to assume that the reading would fall within the following ranges:

32.00-42.00 degrees Celsius or

89.60-107.60 degrees Fahrenheit.

The reading in Celsius scale can always be represented in 4 digits but the reading in Fahrenheit scale may have 4 or 5 digits.

For the purpose of data transmission, one digit can be saved for temperature reading in Fahrenheit scale by subtracting it by 10, i.e. 100.63 Fahrenheit is represented by 90.63. Thus, in one embodiment, the MCU 102 causes the measurements in Fahrenheit scale to be represented by the [measured reading—10 degrees] formula. The decimal point can also be skipped, so the two example readings above can be represented by:

3813 or

9063.

Optionally, a check digit can be added by the MCU 102 to the measured temperature reading after the last digit for protecting the integrity of the data during transmission to the mobile communication device 100, for example:

38136

9063D.

The check digits can be generated by a variety of different algorithms, and it is not important for purposes of this invention which specific algorithm is used. However, the check digit algorithm may expand the character set from decimal (0-9) to hexadecimal (0-F). The 5 digits are then encoded by a channel coding scheme, such as a DTMF scheme employed by the DTMF tone generator 103 seen in FIG. 2, although other schemes may be used.

Table 1 above is used to assign numerical symbols to each combination of frequencies. A dual-tone sinusoidal signal is encoded as a pair of sinusoidal tones from the Table 1 above which are mixed with each other. Each character in the temperature reading is encoded with one frequency picked from a corresponding row and one frequency picked from a corresponding column from the Table 1 above. For example, character “3” is encoded as “f1+f8,” “4” is encoded as “f2+f5.” Tones other than sinusoidal can also be used with one restriction, that the harmonic energy does not affect the detection of the fundamental sinusoidal tone signal.

Each sinusoidal tone encoded above is followed with a silent period that is used to form a space between each successive symbol. The length of each of the tone and silent periods is not important, for example there could be, 100 milliseconds of tone and 50 milliseconds of silent period. Then the reading of “38136” Celsius is encoded and transmitted as an acoustic signal via a buzzer as:

(f1+f8:100 ms) (silent:50 ms) (f3+f5:100 ms) (silent:50 ms) (f1+f6:100 ms) (silent:50 ms) (f1+f8:100 ms) (silent:50 ms) (f2+f7:100 ms) (silent:50 ms).

Essentially, then, the BBT thermometer 101 “calls” the mobile communication device 100 and communicates to it, via the audio channel/microphone input port 104, a set of encoded signals in the form of DTMF tones. The mobile communication device 100 has built-in logic to decode DTMF tones. The mobile communication device 100 then decodes the DTMF tones and communicates the decoded temperature measurement to the APP 111 which records it as numeric data (e.g., 38.13) and analyzes it to check whether it contains a satisfactory reading. If the reading is not satisfactory, the APP 111 prompts the user to take another reading. The APP 111 detects a bad reading based on a set of predetermined characteristics. The APP may characterize a reading as a bad reading if it is abnormally high or low, if it is too far or inconsistent with the trend of the previous readings, or if the thermometer 101 detects a reading that is not stable during the period of measurement. The APP 111 determines whether the reading is a satisfactory reading or a “bad” reading almost concurrently with receiving the reading.

This avoids the need to manually input the thermometer reading into a computer software or application. Such a user may not realize that the reading is a “bad” reading if she manually inputs the data. The APP 111, on the other hand, is able to analyze the trend of the data received in the past and immediately make a determination whether the reading is satisfactory or not. This is particularly advantageous over prior art systems where the user was required to manually input BBT thermometer data, which could often lead to inaccurate BBT data, and, consequently, inaccurate ovulation predictions.

In one embodiment, the APP 111 receives the data from the thermometer 101 on a particular day. The APP 111 inputs the data into a chart and compares it with the data received over a predetermined period preceding that day. The predetermined period could be a few days or weeks. If the data received on a particular day is inconsistent with the trend of the data received over the predetermined period or if the data is inconsistent with the data received on the same day during the previous cycle or cycles, the APP 111 may characterize it as a bad reading. The APP 111 may then prompt the user with a question or a series of questions depending on the character of the reading.

If the reading appears significantly (approximately 0.3 degrees Celsius or more) higher than the trend of the readings over the predetermined period, the APP 111 may prompt the user whether she is feeling sick or feverish or ask the user other supplementary questions. Likewise, the APP 111 may ask the user if she has gone to the bathroom or performed any other activities before using the BBT thermometer. If the user responds that she has, the APP may prompt the user to take a short break, for about 15 minutes, and alarm her to take another measurement afterwards. If the user responds that she has not gone to the bathroom or performed any other activities before taking the BBT thermometer reading, the APP 111 may prompt the user to take a second measurement. If the second measurement is consistent with the first measurement, the APP 111 records the first reading, the second reading, or the average of the two readings as the measurement for that particular day. Taking a second measurement ensures accuracy of the recorded BBT measurements, which in turn increases the accuracy of ovulation predictions provided by the APP 111. Since a variety of different factors may influence the BBT thermometer reading, and since even very slight variations in measurements are significant, it is crucial that each recorded data point is accurate in order to get an accurate ovulation prediction.

If the reading is significantly lower than the trend of the readings over the predetermined period, or if it is significantly lower than the reading received on the same day of the previous cycle or cycles, the APP 111 may prompt the user with another series of predetermined questions, relating to the user's actions before taking the reading and relating to the user's well-being. Depending on the user's response, the APP 111 will likely suggest that the user takes a short break and then alert her to take a second measurement. If the second measurement is consistent with the first measurement, the APP 111 records the first reading, the second reading, or the average of the two readings as the measurement for that particular day.

When all the information is entered, the APP 111 analyzes the data and gives advice to the user in accordance with the data. The analysis is done in real-time and the feedback is provided to the user shortly (i.e., a few seconds or minutes). This eliminates the risk that the user will forget to record the data or that she will record the data incorrectly. The advice may include suggesting that the user employs ovulation test strips to reconfirm the prediction, that the user takes a pregnancy test or schedule a visit to a doctor, or providing an optimal time for intercourse.

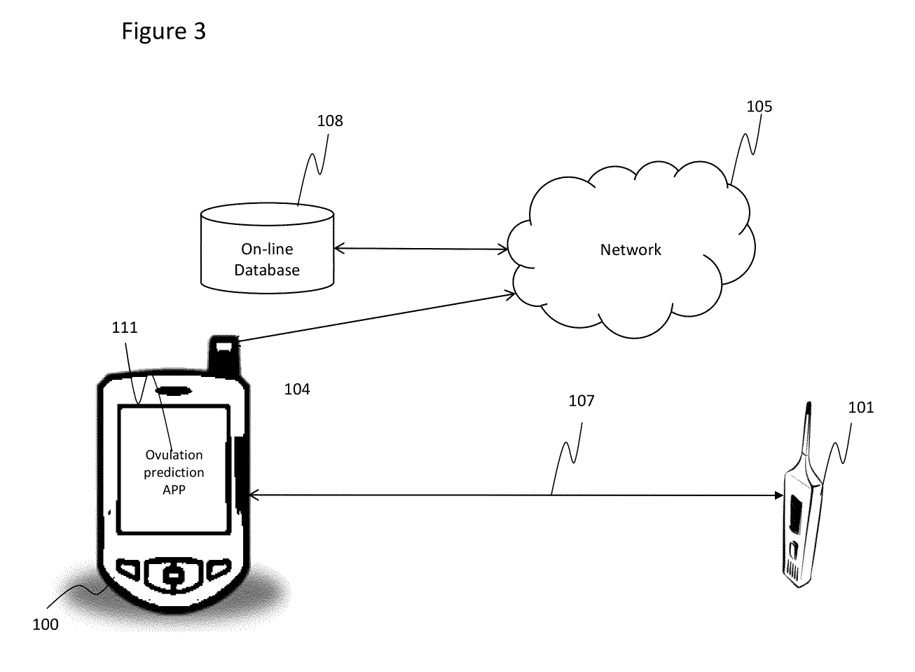

Alternatively, as shown in FIG. 3, the BBT thermometer 101 and the mobile communication device 100 could each be equipped with a Bluetooth or Wi-Fi interface 107 or any other near-field communication technology that allows communication between the thermometer 101 and the smartphone 100. In this case, the thermometer 101 is adapted to transmit the temperature measurements to the mobile communication device 100 directly via the Bluetooth or Wi-Fi connection 107. The thermometer 101 may be adapted to encrypt the temperature measurements before transmission to the mobile communication device 100. The mobile communication device 100 and/or the thermometer 101 may further be equipped with software that allows a Bluetooth, Wi-Fi, or other connection to be established between the devices only if the user inputs a password.

According to yet another embodiment, the BBT thermometer 101 may be adapted to generate sounds during and/or after recordation of temperature data. The smartphone 101 may include a microphone (not shown) and the APP 111 may be adapted to enable the microphone to capture the sounds generated by the thermometer 101.

According to one embodiment, using the APP 111, the user sets an alarm to schedule the measurement of the BBT for the same time every day (preferably in the morning). Other alarms or to-do prompts can also be set to remind the user to record any other supplementary information, such as cervical mucus observations, Luteinizing hormone levels, cervical position, menstrual periods. When the alarm sounds, the user may stop the alarm, for example by touching the screen or a button on the phone. When the alarm is stopped, or when the thermometer 101 is enabled using another command, the APP 111 and the BBT thermometer 101 enter in communication with each other through the connection established with the phone 100, which may be a connection via the audio port 104 as shown in FIG. 2, a Bluetooth or Wi-Fi interface connection 107 as shown in FIG. 3, or any other connection between the devices.

The APP 111 also allows for storage and uploading of analyzed data to a password-protected on-line account, where a chart of the BBT readings and supplementary information can be viewed for on-line consultation at a later date. Such information may also be viewed by a doctor when a user is seeking professional advice.

While particular embodiments and applications of the present invention have been illustrated and described, it is to be understood that the invention is not limited to the precise constructions and compositions disclosed herein and that various modifications, changes, and variations may be apparent from the foregoing descriptions without departing from the spirit and scope of the invention as defined in the appended claims.

1. An ovulation prediction system comprising:
a thermometer coupled to a mobile communication device,
said thermometer being configured to take temperature measurements and including an encoder configured to encode the temperature measurements and transmit the encoded temperature measurements to the mobile communication device;
the mobile communication device including an application for receiving and analyzing said encoded temperature measurements to determine whether the temperature measurements are satisfactory or unsatisfactory based on a set of predetermined characteristics; and
the application being adapted to give feedback to the user in real-time in accordance with the temperature measurement, the application being further adapted to provide an ovulation prediction.

2. The ovulation prediction system of claim 1, wherein the thermometer is coupled to the mobile communication device via an output plug that is coupled to the thermometer and adapted to be inserted into a port of the mobile communication device.

3. The ovulation prediction system of claim 2, wherein the output plug is a headphone plug and the port is a headphone port.

4. The ovulation prediction system of claim 2, wherein the port of the mobile communication device is an audio channel/microphone input port.

5. The ovulation prediction system of claim 1, wherein the thermometer is coupled to the mobile communication device via a Bluetooth, Wi-Fi, or other near-field type connection.

6. The ovulation prediction system of claim 1, wherein the application is further configured to alert a user of the system to take temperature measurements at the same time every day.

7. The ovulation prediction system of claim 1, wherein the mobile communication device is further configured to transmit the analyzed temperature data to an on-line database for storage and retrieval at a later date via one or more communication networks.

8. The ovulation prediction system of claim 1, wherein the mobile communication device is a smartphone.

9. The ovulation prediction system of claim 9, wherein the smartphone is an iPhone®, a Blackberry™, or an Android-based phone.

10. The ovulation prediction system of claim 1, wherein the thermometer is adapted to measure temperature in both Celsius and Fahrenheit units.

11. The ovulation prediction system of claim 1, wherein the thermometer is configured to encode the temperature measurements using a dual-tone multi-frequency tone generator.

12. A method of operating an interactive system for predicting ovulation, comprising the steps of:
coupling, by a user, a thermometer to a mobile communication device;
measuring, by the thermometer, of the user's basal body temperature;
encoding, by the thermometer, of the user's basal body temperature into a format that the mobile communication device is configured to decode;
transmitting, by the thermometer, of the encoded basal body temperature measurement, to the mobile communication device;
receiving and decoding, by the mobile communication device, the encoded basal body temperature measurement;
communicating, by the mobile communication device, to an application integrated within the mobile communication device, the decoded basal body temperature measurement;
analyzing, by the application, of the decoded basal body temperature measurement to determine whether the measurement is satisfactory or unsatisfactory; and
providing feedback and an ovulation prediction to the user in real-time.

13. The method of claim 12, wherein the thermometer is coupled to the mobile communication device via an output plug that is coupled to the thermometer and adapted to be inserted into a port of the mobile communication device.

14. The ovulation prediction system of claim 12, wherein the port of the mobile communication device is an audio channel/microphone input port.

15. The method of claim 12, wherein the user does not manually enter the measured basal body temperature data.

16. The method of claim 12, wherein the thermometer is coupled to the mobile communication device via a Bluetooth, Wi-Fi, or other near-field type connection.

17. The method of claim 12, wherein the application is further configured to alert a user of the system to take basal body temperature measurements at the same time every day.

18. The method of claim 12, wherein the mobile communication device is further configured to transmit the analyzed basal body temperature data to an on-line database for storage and retrieval at a later date via one or more communication networks.

19. The method of claim 12, wherein the mobile communication device is a smartphone.

20. The method of claim 12, wherein the thermometer is configured to encode the basal body temperature measurements using a dual-tone multi-frequency tone generator.
